(19) United States
(12) Patent Application Publication
(10) Pub. No.: US 2009/0221626 A1
(43) Pub. Date: Sep. 3, 2009

(54) AEROSOL FORMULATION FOR INHALATION CONTAINING AN ANTICHOLINERGIC AGENT

(75) Inventor: Friedrich Schmidt, Ingelheim (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 12/094,196

(22) PCT Filed: Nov. 13, 2006
(86) PCT No.: PCT/EP2006/068396

§ 371 Date: Oct. 15, 2008

(30) Foreign Application Priority Data
Nov. 24, 2005 (DE) 10 2005 055 958.1

(51) Int. Cl. A61K 31/46; A61P 11/00

(52) U.S. Cl. 514/291

(57) ABSTRACT

The invention relates to specific aqueous aerosol formulations that are devoid of propellant, containing one or more anticholinergic agents of formula (1), in which X represents an anion, and containing at least one pharmacologically compatible organic acid and optionally additional pharmacologically compatible adjuvants and/or complexing agents. The cation of formula (1′) is contained in the preparation at a concentration of between 411 and 460.32 mg per 100 ml of medicament preparation.

The present invention relates to specific propellant-free, aqueous aerosol formulations containing one or more anticholinergics of formula 1

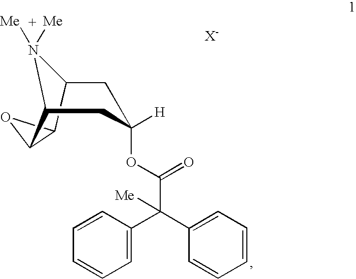

wherein
X⁻ denotes an anion,

at least one pharmacologically acceptable organic acid and optionally other pharmacologically acceptable excipients and/or complexing agents,

wherein the cation of formula 1′

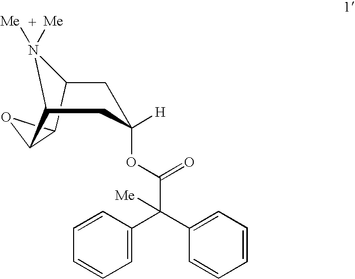

is present in the preparation in a concentration of 411 to 460.32 mg per 100 ml of pharmaceutical preparation.

The compounds of formula 1 are known from WO 02/32899. They have valuable pharmacological properties and as highly effective anticholinergics may provide a therapeutic benefit in the therapy of respiratory complaints, particularly in the therapy of inflammatory and/or obstructive respiratory complaints, particularly for the treatment of asthma or COPD (chronic obstructive pulmonary disease).

The present invention relates to liquid active substance formulations of these compounds which can be administered by inhalation; the liquid formulations according to the invention have to meet high quality standards. The formulations according to the invention may be inhaled orally or nasally. To achieve an optimum distribution of active substances in the lung it makes sense to use a liquid formulation without propellant gases administered using suitable inhalers. Such a formulation may be inhaled both by oral and by nasal route. Those inhalers which are capable of nebulising a small amount of a liquid formulation in the dosage needed for therapeutic purposes within a few seconds into an aerosol suitable for therapeutic inhalation are particularly suitable. Within the scope of the invention, preferred nebulisers are those in which an amount of less than 100 microlitres, preferably less than 50 microlitres, most preferably less than 20 microlitres of active substance solution can be nebulised preferably in one puff or two puffs to form an aerosol having an average particle size of less than 20 microns, preferably less than 10 microns, so that the inhalable part of the aerosol already corresponds to the therapeutically effective quantity.

An apparatus of this kind for the propellant-free administration of a metered amount of a liquid pharmaceutical composition for inhalation is described in detail for example in International Patent Application WO 91/14468 “Atomizing Device and Methods” and also in WO 97/12687, cf. FIGS. 6a and 6b and the accompanying description. In a nebuliser of this kind a pharmaceutical solution is converted by means of a high pressure of up to 500 bar into an aerosol destined for the lungs, which is sprayed. Within the scope of the present specification reference is expressly made to the entire contents of the literature mentioned above.

In inhalers of this kind the formulations of solutions are stored in a reservoir. It is essential that the active substance formulations used are sufficiently stable when stored and at the same time are such that they can be administered directly, if possible without any further handling, in accordance with their medical purpose. Moreover, they must not contain any ingredients which might interact with the inhaler in such a way as to damage the inhaler or the pharmaceutical quality of the solution or of the aerosol produced.

To nebulise the solution a special nozzle is used as described for example in WO 94/07607 or WO 99/16530. Reference is expressly made here to both these publications.

WO 04/022052 A1 also describes aqueous, propellant-free aerosol formulations for the anticholinergic of formula 1. These aqueous formulations contain the anticholinergic of formula 1 in combination with at least one organic or inorganic, pharmacologically acceptable acid and optionally with other pharmacologically acceptable excipients and/or complexing agents.

The problem of the present invention is to provide an aqueous formulation of the compound of formula 1 that meets the high standards needed in order to be able to achieve optimum nebulisation of a solution using the inhalers mentioned hereinbefore and having improved properties compared with the aqueous formulations according to the prior art. The active substance formulations according to the invention must also be of sufficiently high pharmaceutical quality, i.e. they should be pharmaceutically stable over a storage time of some years, preferably at least one year, more preferably two years.

These propellant-free formulations of solutions must also be capable of being nebulised under pressure using an inhaler, the composition delivered by the aerosol produced falling reproducibly within a specified range.

The problem according to the invention is solved by an aqueous pharmaceutical preparation for inhalation containing one or more, preferably one compound of formula 1,

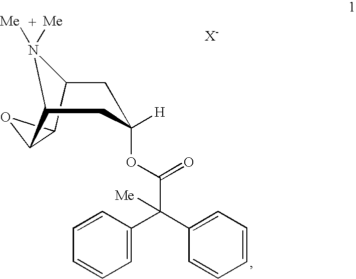

wherein X⁻ denotes an anion, a pharmacologically acceptable organic acid as well as further pharmacologically acceptable excipients and/or complexing agents, while the cation of formula 1′

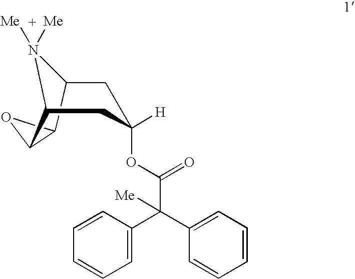

is present in the preparation in a concentration of 411 to 460.32 mg per 100 ml of pharmaceutical preparation.

Within the scope of the present invention it is preferable to use those compounds of formula 1 wherein the anion X⁻ is selected from among the chloride, bromide, iodide, sulphate, phosphate, methanesulphonate, nitrate, maleate, acetate, citrate, fumarate, tartrate, oxalate, succinate, benzoate and p-toluenesulphonate.

Preferably, the salts of formula 1 are used wherein X⁻ denotes an anion selected from among chloride, bromide, 4-toluenesulphonate and methanesulphonate.

Particularly preferred within the scope of the present invention are those formulations which contain the compound of formula 1 wherein X⁻ denotes bromide.

It is preferable to use aqueous pharmaceutical preparations for inhalation containing one or more, preferably one compound of formula 1 wherein X⁻ denotes bromide, a pharmacologically acceptable organic acid as well as further pharmacologically acceptable excipients and/or complexing agents, 100 ml of pharmaceutical preparation containing 500 to 560 mg of the bromide of formula 1.

References to the compound of formula 1 always include within the scope of the present invention all possible amorphous and crystalline modifications of this compound. References to the compound of formula 1 also include within the scope of the present invention all possible solvates and hydrates that may be formed from this compound.

Any reference made within the scope of the present invention to the compound 1′ is to be taken as a reference to the pharmacologically active cation of the following formula

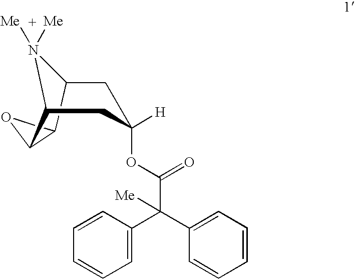

contained in the salts 1.

In the formulation according to the invention the compound 1 is dissolved in water. Co-solvents may optionally be used. However, it is preferable according to the invention not to use an additional solvent.

The concentration of the compound of formula 1 based on the amount of pharmacologically active cation 1′ in the pharmaceutical preparation according to the invention is preferably about 419.2 to 452.1 mg per 100 ml according to the invention. Particularly preferably, 100 ml of the formulations according to the invention contain about 427.44 to 448 mg of 1′, particularly about 434.69 mg of 1′.

If the compound of formula 1 used is the particularly preferred compounds according to the invention wherein X⁻ denotes the bromide, the amount of 1 according to the invention is preferably about 510 to 550 mg of pharmaceutical preparation. Particularly preferably 100 ml of the pharmaceutical preparation according to the invention contain 520 to 545 mg, particularly about 528.821 mg of the compounds of formula 1 .

According to the invention the formulation preferably contains only a single salt of formula 1. However, the formulation may also contain a mixture of different salts of formula 1 .

The pH of the formulation according to the invention is preferably, according to the invention, in the range from 2.5 and 6.5, preferably in the range from 3.0 to 5.0, more preferably in the range from 3.5 to 4.5, particularly in the range from 3.6 to 4.4.

The pH is adjusted by the addition of organic, pharmacologically acceptable acids. Examples of organic, pharmacologically acceptable acids are selected from among ascorbic acid, citric acid, malic acid, tartaric acid, maleic acid, succinic acid, fumaric acid, acetic acid, formic acid and propionic acid. Preferred organic pharmacologically acceptable acids are ascorbic acid, fumaric acid and citric acid, while citric acid is particularly preferred according to the invention. If desired, mixtures of the abovementioned acids may also be used, particularly in the case of acids which have other properties in addition to their acidifying properties, e.g. those which act as flavourings or antioxidants, such as for example citric acid or ascorbic acid.

If desired, pharmacologically acceptable bases may be used to titrate the pH precisely. Suitable bases include for example alkali metal hydroxides and alkali metal carbonates. The preferred alkali ion is sodium. If bases of this kind are used, care must be taken to ensure that the resulting salts, which are then contained in the finished pharmaceutical formulation, are pharmacologically compatible with the abovementioned acid.

Preferably the formulations according to the invention contain, as the organic, pharmacologically acceptable acid, citric acid in a concentration of 2 to 5 mg per 100 ml solution, particularly in a concentration of 3 mg per 100 ml solution.

The formulations according to the invention may contain complexing agents as other ingredients. By complexing agents are meant within the scope of the present invention molecules which are capable of entering into complex bonds. Preferably, these compounds should have the effect of complexing cations, most preferably metal cations. The formulations according to the invention preferably contain editic acid (EDTA) or one of the known salts thereof, e.g. sodium EDTA or disodium EDTA dihydrate (sodium edetate), as complexing agent. Preferably, sodium edetate is used, optionally in the form of its hydrates, more preferably in the form of its dihydrate. If complexing agents are used within the formulations according to the invention, their content is preferably in the range from 5 to 20 mg per 100 ml, more preferably in the range from 7 to 15 mg per 100 ml of the formulation according to the invention. Preferably, the formulations according to the invention contain a complexing agent in an amount of about 9 to 12 mg per 100 ml, more preferably about 10 mg per 100 ml of the formulation according to the invention.

The remarks made concerning sodium edetate also apply analogously to other possible additives which are comparable to EDTA or the salts thereof, which have complexing properties and can be used instead of them, such as for example nitrilotriacetic acid and the salts thereof.

Other pharmacologically acceptable adjuvants may also be added to the formulation according to the invention. By adjuvants and additives are meant, in this context, any pharmacologically acceptable and therapeutically useful substance which is not an active substance, but can be formulated together with the active substance in the pharmacologically suitable solvent, in order to improve the qualities of the active substance formulation. Preferably, these substances have no pharmacological effects or no appreciable or at least no undesirable pharmacological effects in the context of the desired therapy. The adjuvants and additives include, for example, stabilisers, antioxidants and/or preservatives which prolong the shelf life of the finished pharmaceutical formulation, as well as flavourings, vitamins and/or other additives known in the art. The additives also include pharmacologically acceptable salts such as sodium chloride, for example.

The preferred excipients include antioxidants such as ascorbic acid, for example, provided that it has not already been used to adjust the pH, vitamin A, vitamin E, tocopherols and similar vitamins or provitamins occurring in the human body.

Preservatives can be added to protect the formulation from contamination with pathogenic bacteria. Suitable preservatives are those known from the prior art, particularly benzalkonium chloride or benzoic acid or benzoates such as sodium benzoate in the concentration known from the prior art. Preferably, benzalkonium chloride is added to the formulation according to the invention. The amount of benzalkonium chloride is between 1 mg and 50 mg per 100 ml of formulation, preferably about 7 to 15 mg per 100 ml, more preferably about 9 to 12 mg per 100 ml of the formulation according to the invention.

Preferred formulations contain only benzalkonium chloride, sodium edetate and the acid needed to adjust the pH, preferably hydrochloric acid, in addition to the solvent water and the compounds of formula 1 .

The pharmaceutical formulations according to the invention containing compounds of formula 1 are preferably used in an inhaler of the kind described hereinbefore in order to produce the propellant-free aerosols according to the invention. At this point we should once again expressly mention the patent documents described hereinbefore, to which reference is hereby made.

As described at the beginning, a further developed embodiment of the preferred inhaler is disclosed in WO 97/12687 (cf. in particular FIGS. 6a and 6b and the associated passages of description). This nebuliser (Respimat®) can advantageously be used to produce the inhalable aerosols according to the invention. Because of its cylindrical shape and handy size of less than 9 to 15 cm long and 2 to 4 cm wide, the device can be carried by the patient at all times. The nebuliser sprays a defined volume of the pharmaceutical formulation out through small nozzles at high pressures, so as to produce inhalable aerosols.

The preferred atomiser essentially consists of an upper housing part, a pump housing, a nozzle, a locking clamp, a spring housing, a spring and a storage container, characterised by
a pump housing fixed in the upper housing part and carrying at one end a nozzle body with the nozzle or nozzle arrangement,
a hollow piston with valve body,
a power take-off flange in which the hollow body is fixed and which is located in the upper housing part,
a locking clamping mechanism located in the upper housing part,
a spring housing with the spring located therein, which is rotatably mounted on the upper housing part by means of a rotary bearing,
a lower housing part which is fitted onto the spring housing in the axial direction.
The hollow piston with valve body corresponds to a device disclosed in WO 97/12687. It projects partially into the cylinder of the pump housing and is disposed to be axially movable in the cylinder. Reference is made particularly to FIGS. 1-4—especially FIG. 3—and the associated passages of description in the abovementioned International Patent Application. At the moment of release of the spring the hollow piston with valve body exerts, at its high pressure end, a pressure of 5 to 60 Mpa (about 50 to 600 bar), preferably 10 to 60 Mpa (about 100 to 600 bar) on the fluid, the measured amount of active substance solution. Volumes of 10 to 50 microlitres are preferred, volumes of 10 to 20 microlitres are more preferable, whilst a volume of 10 to 15 microlitres per actuation is particularly preferred.

The valve body is preferably mounted at the end of the hollow piston which faces the nozzle body.

The nozzle in the nozzle body is preferably microstructured, i.e. produced by micro-engineering. Microstructured nozzle bodies are disclosed for example in WO-99/16530; reference is hereby made to the contents of this specification, especially FIG. 1 and the associated description.

The nozzle body consists for example of two sheets of glass and/or silicon securely fixed together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns and the length being 7 to 9 microns.

If there is a plurality of nozzle openings, preferably two, the directions of spraying of the nozzles in the nozzle body may run parallel to each other or may be inclined relative to one another in the direction of the nozzle opening. In the case of a nozzle body having at least two nozzle openings at the outlet end, the directions of spraying may be inclined relative to one another at an angle of 20 degrees to 160 degrees, preferably at an angle of 60 to 150 degrees, most preferably 80 to 100°.

The nozzle openings are preferably arranged at a spacing of 10 to 200 microns, more preferably at a spacing of 10 to 100 microns, still more preferably 30 to 70 microns. A spacing of 50 microns is most preferred.

The directions of spraying therefore meet in the region of the nozzle openings.

As already mentioned, the liquid pharmaceutical preparation hits the nozzle body at an entry pressure of up to 600 bar, preferably 200 to 300 bar and is atomised through the nozzle openings into an inhalable aerosol. The preferred particle sizes of the aerosol are up to 20 microns, preferably 3 to 10 microns.

The locking clamping mechanism contains a spring, preferably a cylindrical helical compression spring as a store for the mechanical energy. The spring acts on the power take-off flange as a spring member the movement of which is determined by the position of a locking member. The travel of the power take-off flange is precisely limited by an upper stop and a lower stop. The spring is preferably tensioned via a stepping-up gear, e.g. a helical sliding gear, by an external torque which is generated when the upper housing part is turned relative to the spring housing in the lower housing part. In this case, the upper housing part and the power take-off flange contain a single- or multi-speed spline gear.

The locking member with the engaging locking surfaces is arranged in an annular configuration around the power take-off flange. It consists for example of a ring of plastics or metal which is inherently radially elastically deformable. The ring is arranged in a plane perpendicular to the axis of the atomiser. After the locking of the spring, the locking surfaces of the locking member slide into the path of the power take-off flange and prevent the spring from being released. The locking member is actuated by means of a button. The actuating button is connected or coupled to the locking member. In order to actuate the locking clamping mechanism the actuating button is moved parallel to the annular plane, preferably into the atomiser, and the deformable ring is thereby deformed in the annular plane. Details of the construction of the locking clamping mechanism are described in WO 97/20590.

The lower housing part is pushed axially over the spring housing and covers the bearing, the drive for the spindle and the storage container for the fluid.

When the atomiser is operated, the upper part of the housing is rotated relative to the lower part, the lower part taking the spring housing with it. The spring meanwhile is compressed and biased by means of the helical sliding gear, and the clamping mechanism engages automatically. The angle of rotation is preferably a whole-number fraction of 360 degrees, e.g. 180 degrees. At the same time as the spring is tensioned, the power take-off component in the upper housing part is moved along by a given amount, the hollow piston is pulled back inside the cylinder in the pump housing, as a result of which some of the fluid from the storage container is sucked into the high pressure chamber in front of the nozzle.

If desired, a plurality of replaceable storage containers containing the fluid to be atomised can be inserted in the atomiser one after another and then used. The storage container contains the aqueous aerosol preparation according to the invention.

The atomising process is initiated by gently pressing the actuating button. The clamping mechanism then opens the way for the power take-off component. The biased spring pushes the piston into the cylinder in the pump housing. The fluid emerges from the nozzle of the atomiser in the form of a spray. Further details of the construction are disclosed in PCT applications WO 97/12683 and WO 97/20590, to which reference is hereby made.

The components of the atomiser (nebuliser) are made of a material suitable for their function. The housing of the atomiser and—if the function allows—other parts as well are preferably made of plastics, e.g. by injection moulding. For medical applications, physiologically acceptable materials are used.

FIGS. 6a/b of WO 97/12687 show the Respimat® nebuliser with which the aqueous aerosol preparations according to the invention can advantageously be inhaled.

FIG. 6a shows a longitudinal section through the atomiser with the spring under tension, FIG. 6b shows a longitudinal section through the atomiser with the spring released.

The upper housing part (51) contains the pump housing (52), on the end of which is mounted the holder (53) for the atomiser nozzle. In the holder is the nozzle body (54) and a filter (55). The hollow piston (57) fixed in the power take-off flange (56) of the locking clamping mechanism projects partly into the cylinder of the pump housing. At its end the hollow piston carries the valve body (58). The hollow piston is sealed off by the gasket (59). Inside the upper housing part is the stop (60) on which the power take-off flange rests when the spring is relaxed. Located on the power take-off flange is the stop (61) on which the power take-off flange rests when the spring is under tension. After the tensioning of the spring, the locking member (62) slides between the stop (61) and a support (63) in the upper housing part. The actuating button (64) is connected to the locking member. The upper housing part ends in the mouthpiece (65) and is closed off by the removable protective cap (66).

The spring housing (67) with compression spring (68) is rotatably mounted on the upper housing part by means of the snap-fit lugs (69) and rotary bearings. The lower housing part (70) is pushed over the spring housing. Inside the spring housing is the replaceable storage container (71) for the fluid (72) which is to be atomised. The storage container is closed off by the stopper (73), through which the hollow piston projects into the storage container and dips its end into the fluid (supply of active substance solution).

The spindle (74) for the mechanical counter is mounted on the outside of the spring housing. The drive pinion (75) is located at the end of the spindle facing the upper housing part. On the spindle is the slider (76).

The nebuliser described above is suitable for nebulising the aerosol preparations according to the invention to form an aerosol suitable for inhalation.

If the formulation according to the invention is nebulised using the method described above (Respimat®), the mass expelled, in at least 97%, preferably at least 98% of all the actuations of the inhaler (puffs), should correspond to a defined quantity with a range of tolerance of not more than 25%, preferably 20% of this quantity. Preferably, between 5 and 30 mg, more preferably between 5 and 20 mg of formulation are delivered as a defined mass per puff.

However, the formulation according to the invention can also be nebulised using inhalers other than those described above, for example jet-stream inhalers.

If the pharmaceutical preparation of formula 1 according to the invention, wherein X-denotes bromide, is nebulised using the Respimat®, preferably 47.28 μg to 52.95 μg of the compound of formula 1 are administered per puff (=per actuation of the inhaler). Depending on the desired therapeutic effect, up to 4, preferably up to 3, particularly preferably 1 or 2 actuations of the inhaler (=puffs) may be carried out for each application of the solutions according to the invention.

The present invention also relates to an inhalation kit consisting of one of the pharmaceutical preparations according to the invention described above and an inhaler suitable for nebulising this pharmaceutical preparation. The present invention preferably relates to an inhalation kit consisting of one of the pharmaceutical preparations according to the invention described above and the Respimat® inhaler described above.

The examples of formulations given below serve as illustrations without restricting the subject matter of the present invention to the compositions shown by way of example.

### I. FORMULATION EXAMPLE

100 ml of a particularly preferred pharmaceutical preparation contain the following ingredients, in purified water or water for injections, with a density of 1.00 g/cm³, at a temperature of 15° C. to 31° C.:

| 1 (1′bromide) | | | | disodium | citric |
|---|---|---|---|---|---|
| (μg per dose, | | 1 (1′- | benzalkonium | edetate | acid |
| 2 puffs in | 1 (1′-bromide) | bromide) | chloride | dihydrate | (mg/ |
| each case) | (in mg/100 ml) | (in %) | (mg/100 ml) | (mg/100 ml) | 100 ml) |
| 100.00 | 528.821 | 0.437 | 12 | 12 | 3 |
| 94.55 | 500.000 | 0.413 | 10 | 10 | 3 |
| 100.00 | 528.821 | 0.437 | 10 | 10 | 3 |
| 105.90 | 560.000 | 0.463 | 10 | 10 | 3 |
| 100.00 | 528.821 | 0.437 | 8 | 8 | 5 |

In one possible embodiment a dose to be administered comprises two actuations of the inhaler, i.e. two puffs. Consequently, with the particularly preferred pharmaceutical preparations mentioned above, a total of approx. 94.55 to 105.90 μg, particularly 100 μg of the compound of formula 1 are administered per patient dose. The solutions are preferably used in the Respimat in 4.5 ml cartridges.

1. An aqueous pharmaceutical composition for inhalation containing a compound of formula 1 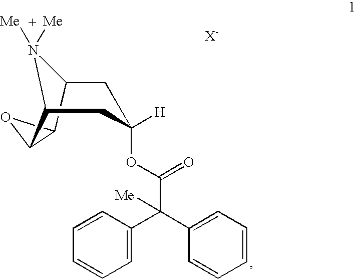
wherein X⁻ denotes an anion;
at least one pharmacologically acceptable organic acid; optionally at least one pharmacologically acceptable excipient, at least one complexing agent or combination thereof; and
wherein the cation of formula 1′ 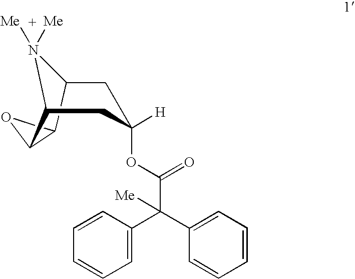
is present in the composition in a concentration of 411 to 460.32 mg per 100 ml of pharmaceutical preparation.

2. The aqueous pharmaceutical composition according to claim 1, wherein the anion X⁻ is selected from chloride, bromide, iodide, sulphate, phosphate, methanesulphonate, nitrate, maleate, acetate, citrate, fumarate, tartrate, oxalate, succinate, benzoate and p-toluenesulphonate.

3. The aqueous pharmaceutical composition according to claim 2, wherein X⁻ is selected from among chloride, bromide, 4-toluenesulphonate and methanesulphonate.

4. The aqueous pharmaceutical composition according to claim 3, wherein X⁻ denotes bromide.

5. The aqueous pharmaceutical composition according to claim 4, wherein formula 1 is present in the composition in a concentration of 510 to 550 mg per 100 ml.

6. The aqueous pharmaceutical composition according to claim 4, wherein formula 1 is present in the composition in a concentration of 520 to 545 mg per 100 ml.

7. The aqueous pharmaceutical composition according to claim 1, wherein the pharmacologically acceptable organic acid is selected from among ascorbic acid, citric acid, malic acid, tartaric acid, maleic acid, succinic acid, fumaric acid, acetic acid, formic acid and propionic acid.

8. The aqueous pharmaceutical composition according to claim 1, wherein the pharmacologically acceptable organic acid is citric acid or ascorbic acid.

9. The aqueous pharmaceutical composition according to claim 8, wherein the citric acid is in a concentration of 2 to 5 mg per 100 ml.

10. The aqueous pharmaceutical composition according to claim 1, wherein the composition has a pH of 3.0 to 5.0.

11. The aqueous pharmaceutical composition according to claim 1, wherein the composition has a pH of 3.6 to 4.4.

12. The aqueous pharmaceutical composition according to claim 1, wherein the pharmacologically acceptable excipient is benzalkonium chloride.

13. The aqueous pharmaceutical composition according to claim 12, wherein the benzalkonium chloride is in a concentration of 7 to 15 mg per 100 ml.

14. (canceled)

15. The aqueous pharmaceutical composition according to claim 1, wherein the complexing agent is editic acid (EDTA) or one of the salts or hydrates thereof.

16. The aqueous pharmaceutical composition according to claim 15, wherein the complexing agent is disodium edetate or one of the hydrates thereof in a concentration of 7 to 15 mg per 100 ml preparation.

17. A method for treatment of respiratory complaints in a patient which comprises administering to a patient in need thereof a pharmaceutical composition according to claim 1.

18. The method of claim 17, wherein 94.55 μg to 105.9 μg of a compound of formula 1, wherein X− denotes bromides is administered.
